(19) United States
(12) Patent Application Publication
(10) Pub. No.: US 2026/0174964 A1
(43) Pub. Date: Jun. 25, 2026

(54) RESERVOIR UNIT FOR INJECTION DEVICE

(71) Applicant: Ypsomed AG, Burgdorf (CH)

(72) Inventors: Leoni Etter, Bern (CH); Stefan Lindegger, Huttwil (CH); Florian Buechi, Muri bel Bern (CH); Manuel Hulliger, Gerlafingen (CH); Marcel Allenspach, Burgdorf (CH); Dominik Zumstein, Lyss (CH); Simon Schuepbach, Burgdorf (CH); Urs Kloetzli, Oberburg (CH); Patrick Hostettler, Hasle (CH); Simon Scheurer, Bern (CH)

(21) Appl. No.: 19/545,557

(22) Filed: Feb. 20, 2026

Related U.S. Application Data
(63) Continuation of application No. PCT/EP2024/071836, filed on Aug. 14, 2024.

(30) Foreign Application Priority Data
Sep. 7, 2023 (EP) 23195978.4

(51) Int. Cl. A61M 5/20; A61M 5/28; A61M 5/315; A61M 5/32

(52) U.S. Cl. CPC A61M 5/20; A61M 5/281; A61M 5/31578; A61M 5/3202; A61M 5/3245; A61M 2005/206; A61M 2005/208; A61M 2005/3247; A61M 2005/3254; A61M 2202/0007; A61M 2202/04; A61M 2205/12; A61M 2205/276

(57) ABSTRACT

A disposable reservoir unit configured for releasable attachment to a reusable drive unit of an injection device includes a reservoir holder for holding a reservoir, two radially extending and oppositely arranged protrusions for releasably attaching the reservoir unit to the drive unit, and a needle cover sleeve guided by guiding means and movable along the longitudinal axis relative to the reservoir holder between a covering position and a retracted position. In a plane perpendicular to the longitudinal axis the needle cover sleeve has a first outer dimension along a first axis and a second outer dimension along a second axis perpendicular to the first axis, the first outer dimension is larger than the second outer dimension, and the protrusions are arranged on the first axis.

### CROSS-REFERENCE TO RELATED APPLICATIONS

This application is a continuation of International Patent Application No. PCT/EP2024/072836, filed Aug. 14, 2024, which in turn claims priority to European Patent Application No. 23195978.4, filed Sep. 7, 2023, each of which is incorporated by reference herein, in their entirety and for all purposes.

### TECHNICAL FIELD

The present disclosure relates to drug delivery devices or medicament delivery devices for dispensing substances and departs from a disposable reservoir unit for an injection device for dispensing a liquid drug through an injection needle by including a reservoir unit configured for releasable attachment to a reusable drive unit of the injection device.

### BACKGROUND

A variety of diseases exist that require regular treatment by subcutaneous administration of a medicament, and a number of drug delivery devices have been developed to support a patient in accurately and controllably delivering an amount of drug in a self-administration process. Delivery devices include injection devices that are removed from the injection site after each medication event or drug delivery process, as well as infusion devices with a cannula or needle that remains in the skin of the patient for a prolonged period of time.

By way of example, diabetes may be treated by self-administration of insulin or its derivatives with the help of multi-variable-dose insulin injection pens. An injection pen device generally has an elongate device body defining a longitudinal main device axis. An automatic injection device has a motor or a drive spring for biasing a plunger rod and shifting a piston in a container barrel, wherein the drive spring may have to be charged or strained manually prior to injection of a dose. A manually powered delivery drive requires a user to manually provide the energy to move the piston, for instance by applying a distal force component to the injection device.

The medicament dose to be injected may typically be manually selected by turning a dosage knob and observing the actual dialed dose from a dose window or display of the insulin pen. A dose is dispensed by inserting the needle into a suited portion of human skin and by moving the piston manually or by pressing a release button of an automatic injection device. Automatic injection devices may include an electronic dose dial mechanism to automatically set a dose.

Also known is the use of autoinjectors with syringes. Autoinjectors usually include a body for housing a syringe as well as a drive mechanism in order to move the plunger of the syringe upon actuation of the autoinjector. The drive mechanism typically includes a source of drive, such as a motor or strong spring for moving a transfer element, for example a rod, which acts on the plunger of the syringe.

For safety and hygiene reasons it is desirable that the needle does not protrude from a housing of the auto injector with the exception of the time when the needle is used for injection of a medicament. Thus, either the autoinjector moves the needle out of the housing for the injection and back into the housing after injection or the housing provides a needle guard which may be moved to unsheathe the needle for injection and which may be moved back to a needle covering position after the injection.

The majority of autoinjectors are configured as single use devices which incorporate both the syringe and the drive mechanism in the same housing. Such devices are usually disposable for hygiene reasons.

Disposable auto injectors including an electric actuator or an electronic control require a source of energy which is usually in the form of a battery. However, in this case, the autoinjectors should not be disposed in the regular waste, but have to be subjected to special disposal or to recycling, which is an additional burden to the patient. Further, disposing a battery, motor and/or electronics after a single use is a waste of resources and increases the costs of the auto injector.

In order to account for a need to handle and dispose the autoinjector parts differently semi-reusable autoinjectors have been developed. Such autoinjectors typically include a reusable drive unit as well as a disposable syringe unit which may be releasably coupled to the drive unit. The drive unit usually includes the drive mechanism and electronics whereas the syringe unit includes the syringe with the needle and a needle cover sleeve. The user can thus discard the syringe unit when it is empty or after use and can load the drive unit with a new syringe unit for the upcoming injection.

WO 21254744 A1 discloses a reusable autoinjector with a syringe unit releasably attachable to a drive unit. The syringe unit includes two oppositely arranged cams in form of protrusion in a proximal end portion of the syringe unit. When the syringe unit is inserted into the drive unit the protrusions travel in respective slots of the drive unit. When the syringe unit is then rotated about the longitudinal axis relatively to the drive unit in order to attach the syringe unit to the base unit, the protrusions engage a respective cam profile.

WO 23275254 A1 discloses a reusable autoinjector where upon loading of the syringe unit into the drive unit, syringe unit securing tabs at the proximal end snap behind corresponding securing openings in the drive unit housing to couple the syringe unit to the drive unit.

EP 4108277 A1 discloses a disposable syringe unit attachable to a reusable drive unit of an reusable autoinjector. The syringe includes two oppositely arranged protrusion in a proximal end portion to attach the syringe unit to the drive unit. Furthermore, the syringe unit includes an opening for locking arms of the drive unit to lock the syringe unit to the drive unit.

### SUMMARY

The present disclosure relates to a disposable reservoir unit for an injection device, in particular for a reusable autoinjector, for dispensing a liquid drug through an injection needle. The reservoir unit is configured for releasable attachment to a reusable drive unit of the injection device. The reservoir unit includes:
a reservoir holder for holding the reservoir in place relative to the drive unit and adapted to be releasably connected to the drive unit and including guiding means and wherein the reservoir holder extends along a longitudinal axis;
two radially extending and oppositely arranged protrusions for releasably attaching the reservoir unit to the drive unit;
a needle cover sleeve guided by the guiding means and movable along the longitudinal axis relative to the reservoir holder between a covering position in which the injection needle is covered and a retracted position in which the injection needle exposes and protrudes from the needle cover sleeve. The needle cover sleeve further includes two oppositely arranged lateral locking openings adapted to accommodate actuation arms of the drive unit to lock the reservoir unit to the drive unit.

Furthermore, in a plane perpendicular to the longitudinal axis the needle cover sleeve has a first outer dimension (or outer diameter) along a first axis and a second outer dimension (or outer diameter) along a second axis perpendicular to the first axis and wherein the first outer dimension is larger than the second outer dimension. The first and second outer dimensions are measured on the outer surface of the needle cover sleeve without the guiding elements, cams or knobs protruding from the outer surface of the needle cover sleeve. The protrusions are arranged on the first axis or/and aligned to the first axis.

The needle cover sleeve further includes two oppositely arranged pairs of guiding elements which protrude from the outer surface of the needle cover sleeve, for example protrude radially and which are adapted to engage counter guiding elements in the reservoir unit to prevent rotation of the reservoir unit relative to the drive unit. The guiding elements of each pair are arranged next or close to the first axis. Namely, a first guiding element of each pair may be arranged on a first side (or left side) and a second guiding element of each pair may be arranged on a second side (or right side) of the first axis in a plane perpendicular to the longitudinal axis.

The second outer dimension of the needle cover sleeve is between 2 and 3.6 times larger than dimension between the first and second guiding element of each pair. The distance between the first and second guiding element is measured from a center of the first guiding element to a center of the second guiding element of each pair in the plane perpendicular to the longitudinal axis.

The second dimension is a maximum of 3.6 times, for example a maximum of 3.2 times larger or longer than the distance between the first and second guiding element of each pair measured in a plane perpendicular to the longitudinal axis.

That means the reservoir unit has a compact design in the plane perpendicular to the longitudinal axis (cross-section) and the relation between the guiding element distance and the second dimension is optimal. Thus, the cross-section is as small as possible and the distance between the guiding elements provides a safe and reliable guiding during attachment of the reservoir unit to the drive unit. Furthermore, the guiding elements spaced apart allow a reliable guiding of a needle cover sleeve movement along the longitudinal axis while maintaining a compact design.

Moreover, the reservoir unit having the protrusion on the first axis with the larger outer dimension allows for an easy and quick attachment to the reservoir unit because the protrusions are located on the outermost portion of the reservoir unit form. The locking openings provide access to the locking arms of the drive unit such that after attachment of the reservoir unit to the drive unit the locking arms can enter into the locking openings to hold and lock the reservoir unit to the drive unit such that the reservoir unit cannot be moved in a distal direction relative to the drive unit. The reservoir unit is thus hold by a form-fit and cannot be removed from the drive unit unless release and retraction of the locking arms.

The protrusions are adapted to engage and to cooperate with corresponding engaging elements such as clamps, hooks or recesses in the drive unit to releasably couple the reservoir unit to the drive unit. Upon insertion of the reservoir unit in an opening in a drive unit housing the engaging element may be deformable or deflectable to engage the protrusion. The protrusions may be adapted to be attached to the drive unit engaging element to form a releasably snap-fit connection. The protrusions may be integrally formed (monolithic) with the reservoir holder or, alternatively, the protrusions may be formed by a separate member of the reservoir unit such as a holding structure or a connecting part.

The reservoir unit has an extension in a plane perpendicular to the longitudinal axis which is larger along the first axis than along the second axis, which is perpendicular to the first axis. That means the reservoir unit has a non-circular cross-section. The outer form or shape of the reservoir unit in a plane perpendicular to the longitudinal axis or a cross-section may be, for example, an oval, a rectangular or any free form. As the dimension is larger along the first axis than along the second axis an opening in the drive unit may have a corresponding non-circular shape such that the outer form may support the user to align the reservoir unit relative to the drive unit before attachment. Furthermore, the non-circular cross-section prevents the reservoir unit from being rolled away if not connected to the drive unit.

The guiding elements provide a safe and reliable process for attachment of the reservoir unit to the drive unit as the guiding elements ensure that the reservoir unit keeps rotationally correctly positioned and aligned relative to the drive unit during attachment. The correct rotational orientation of the reservoir unit to the drive unit is crucial as the drive unit locking arms have to be correctly positioned to the locking openings to be able to be inserted into the openings to lock the reservoir unit to the drive unit. Hence, the guiding elements provide aid to the user to rotationally position the reservoir unit to the drive unit during attachment.

The distance between the first and second guiding element may be between 6.5 and 9 mm and, for example, between 6.9 and 8.3 mm. The dimension or diameter of the needle cover sleeve along the first axis may be between 16 and 23.5 mm (for example between 18 mm and 23 mm) and the dimension or diameter along the second axis may be between 15 and 21 mm (for example between 17 mm and 20.5 mm).

The drive unit may be sleeve-shaped providing an opening to accommodate at least a proximal part of the reservoir unit for attachment. The counter guiding elements may be arranged inside the opening in this case. The guiding elements of the reservoir unit may guide the reservoir unit during the insertion process such that the reservoir unit remains in the correct rotational orientation during insertion and remains in the correct orientation in a final attached position.

In a preferred embodiment the guiding elements have a thickness or width (in a plane parallel to the longitudinal axis) which is thinner than a thickness of the outer sleeve wall of the needle cover sleeve. That means the guiding elements are made of a minimum quantity of material. Unlike known approaches with guiding cam having a massive design the two pairs of thin-walled guiding elements help to reduce the material of the disposable reservoir unit and thus contribute to reduction of waste.

The guiding elements may have, for example, a thickness of less than 1.5 mm and for example less than 1 mm. In a preferred embodiment the thickness of each guiding element is between 0.6 mm and 1.2 mm, for example, between 0.8 mm and 1 mm

By way of example, the guiding elements may be protrusions, knobs, cams, rails or ribs extending along the longitudinal axis. The counter elements in the drive unit may be, for example, grooves, nuts or rails adapted to guide the guiding elements of the reservoir unit along the longitudinal axis during the attachment process.

The disposable reservoir unit may be part of the semi-disposable (also named as semi-reusable) injection device. The reservoir unit is hence not operational without a reusable drive unit of the injection device. To prepare the injection device for an injection the user has to attach or to connect the reservoir unit to a drive unit. The drive unit may include drive means adapted to dispense the liquid drug from the reservoir held inside the reservoir unit once the reservoir unit is attached to the drive unit.

The injection device may be a manual or an automatic injection device. Automatic devices or autoinjectors typically include automatic drive means such as an elastic element (for example a pre-tensioned spring) or an electric motor to drive a dispensing member to dispense the liquid drug form the reservoir. Manual injection devices include a manual drive such as a dispensing button that has to be pressed by the user to move the dispensing member in a dispensing direction.

The reservoir unit includes the two oppositely arranged protrusions. “Oppositely arranged” means the two protrusions are 180° spaced apart from each other in the circumferential direction. Correspondingly, the two locking openings are 180° spaced apart from each other and the two pairs of guiding elements are 180° spaced apart from each other.

The needle cover sleeve is movable along the longitudinal axis and guided by the reservoir holder. The needle cover sleeve is adapted to cover or envelop a needle of the reservoir (prefilled syringe or cartridge with releasably attachable needle) mounted inside the reservoir holder. The movement of the needle cover allows for switching between a safety state in which the needle is covered and the needle does not protrude from a device housing and an injection state in which the needle is uncovered and exposes or protrudes from the housing.

The reservoir holder is adapted to hold the reservoir in place relative to the reservoir holder and for example relative to a drive unit housing. The reservoir holder may have clamping means or stop elements or abutting surfaces to fix and hold the reservoir by form fit or/and by force fit.

The guiding elements may protrude from an outer surface of the needle cover sleeve. The guiding elements protrude along an axis which is angled to the first axis in an angle between 0° and 45°, for example in an angle between 0° and 30° in a plane perpendicular to the longitudinal axis. The guiding elements protruding in the above mentioned range of angles allow a reliable guiding of the reservoir unit relative to the drive unit.

The first outer dimension of the reservoir unit may be between 1.05 and 1.5 time larger than the second outer dimension. This ratio between the first (height) and second (width) dimension and provides a compact design of the reservoir unit.

The dimension along the first axis may be between 16 and 23.5 mm (for example between 18 mm and 23 mm) and the dimension along the second axis may be between 15 and 21 mm (for example between 17 mm and 20.5 mm).

In an example embodiment the needle cover sleeve is the outermost part of the reservoir unit and the reservoir holder is concentrically arranged inside the needle cover sleeve. That means the needle cover sleeve is the outermost part excluding a cap or a rear opening element, if any. That means the reservoir unit does not include any outer housing or shell but only the movable needle cover sleeve. This provides for a simple design.

For example, in a plane perpendicular to the longitudinal axis an outer form or shape of the reservoir unit is a regular oval form which extends along the length of the reservoir unit in the longitudinal direction. For example, the oval form extends along the entire length of the reservoir unit excluding a device cap, if any. That means the reservoir unit (without cap) has just a single cross-section which is the oval form

The oval form prevents the reservoir unit from rolling away if separated from the drive unit. Moreover, the regular oval form allows a label or tag to be applied to an outer surface of the reservoir unit in a simple and cost-effective manner.

Each protrusion may be arranged adjacent to the respective locking opening with respect to the longitudinal direction. That means the holding of the reservoir unit by the protrusions and the locking of the reservoir unit (preventing an unintentional release of the reservoir unit from the drive unit) are close together allowing a compact design of the reservoir unit.

In an example embodiment locking openings are arranged distally from the protrusion or on a distal side of the protrusions. That means the protrusion can be in a proximal portion or at a proximal end of the reservoir unit and thus can reliably hold the reservoir unit in place relative to the drive unit. The actuation arms of the drive unit can be inserted into the locking openings to lock the reservoir. As the openings are distally from the protrusions and hence in a more distal part of the reservoir unit the latter can be held stable in place.

The locking openings may extend along the longitudinal axis such that the needle cover sleeve is not hindered and can be moved relative to the protrusions between the covering position and the retracted position when the actuation arms are positioned inside the locking openings. That means the longitudinally extending openings provide enough room for the actuation arms to be engaged and positioned in the openings while the needle cover sleeve can be moved between its covering and its retracted position. Hence, when the actuation arms are inserted the do not block or hinder the needle cover sleeve movement.

The protrusions may have a rectangular-shaped cross section in a plane parallel to the longitudinal axis. Correspondingly, a counter element (for example a clamp, recess) of the drive unit adapted to engage the protrusions to couple and attach the reservoir unit to the drive unit may have also a rectangular-shaped cross section to accommodate or envelop the protrusion in an attached state.

The needle cover sleeve may include further two oppositely arranged second openings accommodating the protrusions and wherein the protrusions are accessible therethrough. That means the protrusions are located inside the openings and an outermost end (radially outer free end) of the protrusion may be flush with an outer surface of the needle cover sleeve or alternatively the free end of the protrusion may protrude from the needle cover sleeve outer surface. As the protrusions are on the first axis the second openings are correspondingly aligned to the first axis too.

The second openings may extend along the longitudinal axis such that the needle cover sleeve can be moved relative to the protrusions between the covering position and the retracted position without covering the protrusions. That means the protrusion can be engaged with counter elements in the drive unit and the needle cover sleeve does not contact or abut the protrusions or counter elements and hence the needle cover sleeve can be moved between its covering position and its retracted position without being blocked or hindered by the protrusions.

In a preferred embodiment the reservoir unit includes further a lock element movable relative to the needle cover sleeve and wherein the lock element can be in a locking position in which the lock element engages the needle cover sleeve and prevents the needle cover sleeve from being moved out of the covering position. Furthermore, the lock element can be in an unlocking position in which it does not engage the needle cover sleeve such that the needle cover sleeve is not prevented from being moved out of the covering position.

The lock element is a further safety means to prevent unintentional access to the needle and thus may prevent injuries caused by the injection needle. As the lock element can lock the needle cover sleeve in the covering position the injection needle is covered and the user can not (unintentionally) retract the needle cover sleeve. The lock element may be controlled and switched between the locking position and the unlocking position by the drive unit by means of the actuation arm or by another movable member of the drive unit.

The lock element is a radially protruding flexible arm with a free end portion and wherein the needle cover sleeve includes a stop surface and wherein the free end portion is adapted to engage the stop surface to hold the needle cover sleeve in the covering position when the flexible arm is in its locking position. That means the flexible arm is in its locking position when it abuts or contacts the stop surface and the flexible arm is in its unlocking position when it is released from the stop surface and when it does not prevent the needle cover sleeve from being moved out of the covering position.

The end portion may engage the stop surface in a non-deflected position of the arm and the end portion is disengaged from the stop surface in a deflected state of the arm thereby allowing the needle cover sleeve to be moved out of the covering position.

The deflection allows for quick and reliable switching between the locking position and the unlocking position. That means the arm can quickly be switched to engage the stop surface and thus block the needle cover sleeve and deflected to be moved away from the stop surface and thus release the needle cover sleeve.

In an example embodiment the flexible arm is integrally formed in the reservoir holder and monolithic with the reservoir holder. The flexible arm and the reservoir holder may be made by plastic injection molding. That allows a simple manufacturing.

The reservoir holder includes clamping element adapted to be pressed on an outer surface of the reservoir and the reservoir holder further includes a bearing surface inside the reservoir holder adapted to abut a distal shoulder of the reservoir such that the reservoir is non-movable held relative to the reservoir holder. The clamping element and the bearing surface fix and immovably held the reservoir inside the reservoir holder. That means the reservoir does not move before, during and after the injection process.

The reservoir unit without the reservoir may be devoid of metallic parts. That means apart from the reservoir with its reservoir elements (piston, needle, rigid needle shield directly mounted on the needle in a shipping state) the reservoir unit includes exclusively plastic parts and for example the reservoir unit includes exclusively injection molded thermoplastic plastic parts (apart from the reservoir and its reservoir elements). That means the reservoir unit is devoid of any metal parts such as springs, clamps or the like. This facilitates the production and contributes to a cost-efficient design.

The needle cover sleeve may include a machine-readable code attached on a surface of the needle cover sleeve. The code includes a reservoir unit specific identification information.

The code includes reservoir unit specific identification information. An external code reader may read the information from the code and based on read identification information the reservoir unit and the drug inside the reservoir may be identified. The identification information may include data about the reservoir, the drug, a volume, and an expiration date or injection instruction for the drug contained inside the reservoir.

The external code reader may be an external device or it may be in the drive unit and adapted to read the code once the reservoir unit is attached to the drive unit.

The machine-readable code may be implemented in form of optical code or a code readable based on electromagnetic fields. Examples for an optical code are an Object Identifier code (OID), QR codes, bar codes, color codes and optical patterns. Examples for codes based on electromagnetic fields are a radio-frequency identification (RFID) tag or near-field communication (NFC) tags.

The code may be attached on an outside surface of the cover member, for example, by means of a label or sticker including the code. Alternatively, the code may be on an inside surface or integrally formed in a wall of the cover member as electromagnetic tag or chip.

The reservoir unit further includes the reservoir in form of a syringe including a cylindrical barrel made of glass and an injection needle made of steel inseparably connected to the barrel. Alternatively the reservoir of the reservoir unit is a cartridge including a needle mounting portion on its distal end adapted to be releasably connected to a needle prior injection.

The present disclosure further relates to an injection device including the disposable reservoir unit as described above and the reusable drive unit. The latter includes a plunger rod and a drive for moving the plunger rod in a dispensing direction to dispense the drug from the reservoir. The reservoir unit is releasably attachable to the drive unit.

As mentioned above the injection device may be a manual injection device requiring a user force to drive the plunger rod to dispense the drug or alternatively the injection device may be an automatic injection device including an automatic drive, for example, in form of a biased spring member or an electric motor.

The drive unit may further include flexible holding arms engaging the radial extending protrusion of the reservoir unit if the reservoir unit is attached to the drive unit. The flexible holding arms allow for a releasably attachment of the reservoir unit to the drive unit.

In an example embodiment the cover member may be locked to the reservoir holder in the covering position, and the cover member may be unlocked by relative movement between the reservoir unit and the drive unit. The reservoir unit may be moved manually by a user or automatically by the automatic drive of the drive unit.

The injection device may be a semi-reusable (or semi-disposable) autoinjector. The automatic drive may include an electric motor adapted to drive the plunger rod to dispense the drug from the reservoir.

### BRIEF DESCRIPTION OF THE DRAWINGS

FIG. 1 depicts a perspective view of an autoinjector according to the disclosure;

FIG. 2 depicts a perspective view of a syringe unit of the autoinjector;

FIG. 3 depicts an exploded view of the syringe unit;

FIG. 4 depicts an exploded view of a second embodiment of the syringe unit;

FIG. 5 depicts a cross-sectional view of a drive unit of the autoinjector;

FIG. 6a depicts a cross-sectional view of the autoinjector with the syringe unit attached to the drive unit;

FIG. 6b depicts a cross-sectional view of the autoinjector of FIG. 6a wherein the plane for the cross-sectional view is offset and parallel a plane in the center of the longitudinal axis;

FIG. 6c depicts a cross-sectional view of the autoinjector of FIG. 6a wherein the plane is offset and parallel the plane in the center of the longitudinal axis;

FIG. 7 depicts a cross-sectional view of the autoinjector with a syringe unit locked to the drive unit and without cap and a released cover sleeve before an injection;

FIG. 8 depicts a cross-sectional view of the autoinjector after a dispensing of a dose;

FIG. 9a depicts a perspective view of the drive unit without the outer shell parts; and

FIGS. 9b, 9c depict a perspective view of the upper and lower shell part of the drive unit.

### DETAILED DESCRIPTION

### Definitions

In the present context, the terms “substance”, “drug”, “medicament” and “medication” are to be understood to include any flowable medical formulation suitable for controlled administration through a means such as, for example, a cannula or a hollow needle, and comprises a liquid, a solution, a gel or a fine suspension containing one or more medical active ingredients. A medicament can be a composition comprising a single active ingredient or a pre-mixed or co-formulated composition with more than one active ingredient present in a single container. Medication includes drugs such as peptides (e.g., insulin, insulin-containing drugs, GLP-1 containing drugs or derived or analogous preparations), proteins and hormones, active ingredients derived from, or harvested by, biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances

The term “distal” is meant to refer to the direction or the end of the drug delivery device carrying an injection needle or an injection cannula, whereas the term “proximal” is meant to refer to the opposite direction or end pointing away from the needle or cannula. For instance, in the present description the term “distal” refers to the side where the injection needle is located. This is on the left-hand side in the figures. The term “proximal” refers to the opposite side or rear end of the device and is on the right-hand side in the figures.

The term “injection system” or “injector” refers to a device that is removed from the injection site after each medication event or drug delivery process, whereas the term “infusion system” refers to a device with a cannula or needle that remains in the skin of the patient for a prolonged period of time, for example, several hours.

The term “regular” means the oval form is not interrupted, for example, by a flattened portion or by a straight section. The term “oval” form excludes a circular or a rectangular cross-section but the term “oval” includes shapes such as an ellipse or a form that is egg-shaped.

### DESCRIPTION OF THE FIGURES

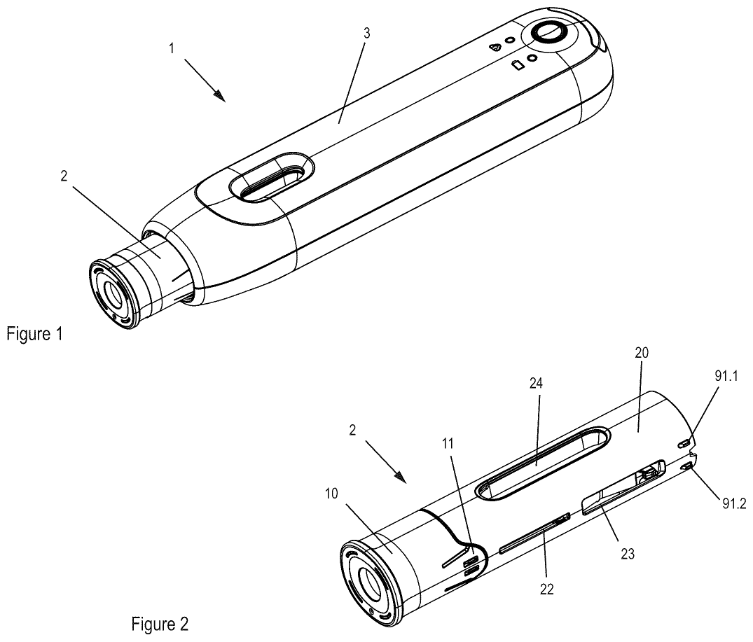

FIG. 1 shows a perspective view of an injection device of a first embodiment in form of a semi-reusable autoinjector 1. The autoinjector 1 includes a disposable reservoir unit in form of a syringe unit 2 and a reusable drive unit 3. As shown in FIG. 1 the drive unit 3 has an elongated housing defining a longitudinal axis and includes a button 4 at its proximal end. The drive unit further includes an opening or cavity adapted to accommodate a proximal portion of the syringe unit 2 to attach or couple the syringe unit 2 to the drive unit 3.

In the following the structural features of the syringe unit 2 and the drive unit 3 will be described in detail. Subsequently, the function of the semi-reusable autoinjector 1 will be explained.

### Syringe Unit

FIG. 2 depicts a perspective view of the syringe unit 2 without the drive unit. The syringe unit 2 has an elliptical form in the cross section. The elliptical form extends along the entire length of the syringe unit in the longitudinal axis. On a distal end the cap 10 is mounted in an initial state or shipping state. The proximal end includes a holding structure as described below to releasably attach the syringe unit 2 to the drive unit 3.

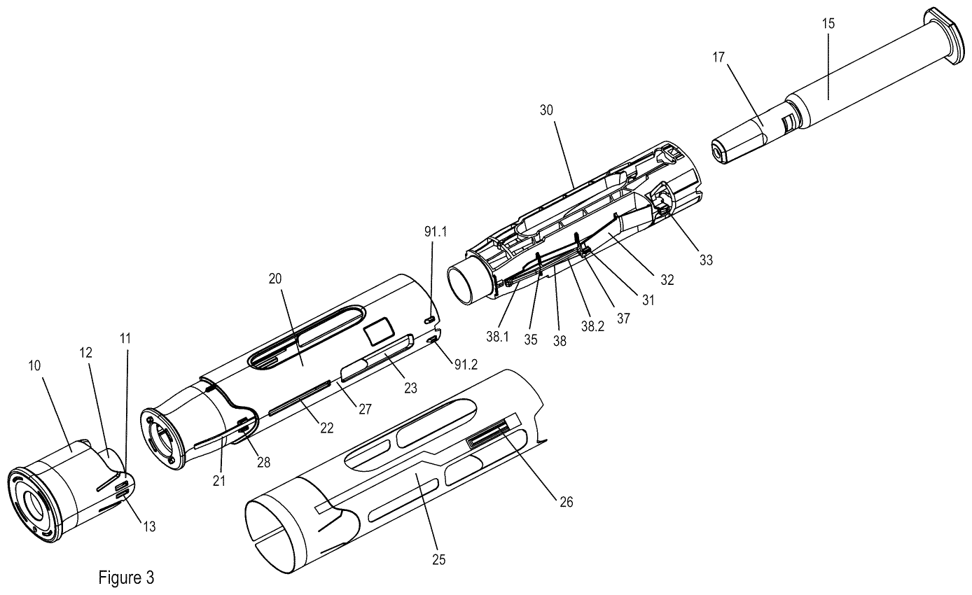

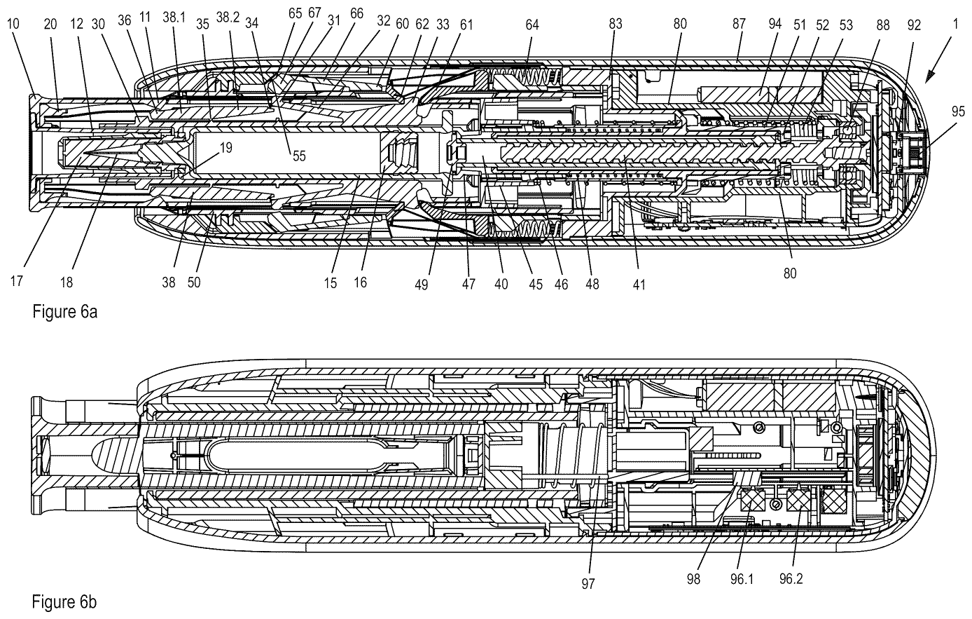

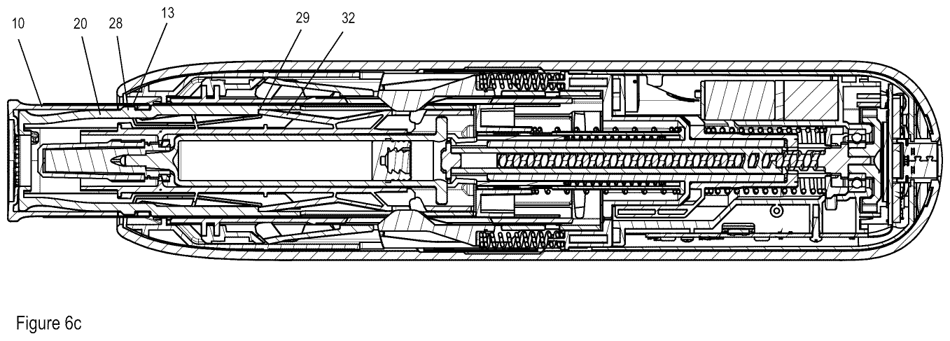

FIG. 3 depicts an exploded view of the syringe unit 2. As shown in FIG. 3 the syringe unit 2 includes a syringe holder 30 (reservoir holder), a needle cover sleeve 20 coaxially arranged around the syringe holder 30 and movable relative thereto along the longitudinal axis, a label 25 wrapped around the needle cover sleeve 20, the cap 10 with a RNS remover and a reservoir in form of a prefilled syringe 15 (FIG. 6a) including a liquid drug and a rigid needle shield 17 (RNS) initially mounted on the injection needle. The syringe holder 30, the cover sleeve 20 and the cap 10 are individually injection-molded plastic parts which are all made of the same material, for example, polypropylene.

The prefilled syringe 15 with a barrel made of glass is rotationally and axially fixed inside the sleeve-shaped syringe holder 30. For that purpose, the syringe holder 30 includes clamping elements (not shown) pressing on an outer surface of the syringe barrel. The syringe holder 30 provides a bearing surface 19 (see FIG. 6a) on its inside adapted to abut a shoulder of the syringe barrel. The syringe holder 30 further features longitudinal guiding rails (not shown) on its outside for the needle cover sleeve 20 such that the needle cover sleeve 20 can be shifted exclusively in the longitudinal direction relative to the syringe holder 30.

As shown in FIGS. 3 and 6a the syringe holder 30 includes two oppositely arranged and in longitudinal direction extending rockers 38 or seesaws. Each rocker 38 is pivotable around a center 35 which is formed by a flexible or elastic connection fixing the rocker 38 on an outer surface of the syringe holder 30. The center 35 divides the rocker 38 into a distal portion 38.1 or distal arm and a proximal portion 38.2 or proximal arm. The distal portion 38.1 is adapted to engage and deflect a cap holding tongue 11. For this purpose, the distal portion 38.1 includes a nose 36 (see FIG. 6a) adapted to engage a projection of the cap holding tongue 11 to disengage a snap-fit connection between tongue 11 and needle cover sleeve 20 or holder 30.

As best shown in FIG. 6a the proximal portion of the rocker 38 includes an actuation surface 34 adapted to be contacted by a free end of a lock element in form of a release arm 32. The rocker 38 is adapted to be tilted or pivoted around the center 35 between a first or initial tilt position where the distal portion 38.1 is in a radially inward position and a second tilt position where the distal portion 38.1 is in a radially outward position.

The two radially deflectable lock elements or release arms 32 (see FIG. 3 or 6a) are arranged on opposite sides of the syringe holder 30 and include each a distally directed free end with an end bulge 31 and a connecting end which connects the release arms 32 to the syringe holder 30. At the free end and lateral to the bulge 31 are two side support surfaces 37 on both sides of the bulge 31 to guide the release arms 32 as descripted below. The rocker 38 and the release arm 32 are integrally formed in the syringe holder 30 as a monolithic part. That means the syringe holder 30 with the rockers and the release arms is injection molded as one single part as best shown in FIG. 3.

In an initial state the release arms 32 are in a non-deflected state and in a locking position and prevent a movement of the cover sleeve 20 relative to the syringe holder 30 in a proximal direction. Hence, the cover sleeve 20 shields or covers the injection needle of the syringe 15 in a covering position. For that purpose, a free end of the locking arms 32 abut stop surfaces (not shown) inside the cover sleeve 20. As provided herein, once the locking arms 32 are deflected radially inwards and are in an unlocking position the cover sleeve 20 can be pushed proximally.

The syringe holder 30 further includes two oppositely arranged and radially protruding end ledges or protrusions 33 at a proximal end of the syringe holder 30 for engagement with clamping arms 61 (FIG. 6a) inside the drive unit 3. The protrusion 33 have a rectangular cross section in a plane parallel to the longitudinal axis and the clamping arms of the drive unit have on their free end a corresponding opening with a rectangular cross section to accommodate and engage the protrusions 33.

As shown in FIG. 3 the needle cover sleeve 20 has a non-circular cross section and in particular an oval form in a cross section or in a plane perpendicular to the longitudinal axis. An outer dimension along a first axis (height) of the needle cover sleeve is between 18 and 23 mm and an outer dimension along a second axis perpendicular to the first axis (width) is between 17 and 20.5 mm in a plane perpendicular to the longitudinal axis. The height is between 1.05 and 1.5 time larger than the width of the needle cover sleeve in the cross-section.

On an outer surface and in a proximal end portion of the needle cover sleeve 20 two pairs of guiding elements 91.1, 91.2 are oppositely arranged. The guiding elements 91.1, 91.2 protrude from the surface of the needle cover sleeve. The guiding elements protrude along an axis which is angled to the fist axis (height) in an angle between 20° and 40° in a plane perpendicular to the longitudinal axis. A dimension from a center of a first guiding element 91.1 to a center of a second guiding element 91.2 of each pair in the plane perpendicular to the longitudinal axis is between 1.9 and 3.6 times shorter or smaller than the width or a diameter along the second axis of the needle cover sleeve 20. Namely, the distance between the first and second guiding element is between 6.9 mm and 8.3 mm and the thickness of each guiding element 91.1, 91.2 is between 0.8 and 1 mm.

As shown in FIGS. 2 and 3 the guiding elements 91.1, 91.2 are implemented in form of small ribs extending along the longitudinal axis. The drive unit includes on an inner side of the opening corresponding guiding grooves guiding the guiding elements 91.1, 91.2 along the longitudinal axis. During the attachment when the proximal part of the syringe unit is inserted into the drive unit the guiding elements thus guide the syringe unit and prevent a rotation relative to the drive unit. Moreover, during retraction and distal movement of the needle cover sleeve 20 relative to the drive unit the guiding elements 91.1, 91.2 guide the needle cover sleeve.

The needle cover sleeve including a medicament window 24 allowing the user to view the fill level of the syringe inside the syringe unit. The needle cover sleeve further includes two oppositely arranged lateral openings 21 for a nose 36 of the rocker 38 to reach the cap holding tongues 11. The cover sleeve 20 further features two oppositely arranged locking openings 22 for access to the release arms 32. The locking openings 22 are located distally from two oppositely arranged openings 23 for access to the protrusions 33 from outside. The locking openings 22 and the openings 23 are best shown in FIG. 2. The locking openings 22 and the openings 23 for the protrusions extend both along the longitudinal axis and have a rectangular shape such that the needle cover sleeve 20 can be moved between its covering position and its retracted position without being hindered by the protrusions 33 and/or by the actuation arms 66 inserted into the locking openings 22.

The label 25 is wrapped around an outer surface of the syringe unit. The label includes a RFID code 26 integrated in the label and positioned in a proximal portion of the label 25. The code 26 includes information about the syringe unit for identification and may include further information about the drug inside the syringe, the volume, expire date and dispensing parameter such as injection speed and holding time. The code 26 may have further country-specific information such as a user language to adjust a user interface language.

At a distal end of the needle cover sleeve 20 the cap 10 is releasably attached by a snap-fit connection. The cap 10 includes on two sides two deflectable holding members in form of holding tongues 11 on each side. The tongues 11 include on the free end an indentation or recess 13 adapted to be snapped to a protrusion 28 arranged on an outer surface of the cover sleeve 20 (also shown in FIG. 6c). Each tongue further includes a lobe projecting in the opening 21 in the needle cover sleeve if the cap is mounted. The release of the snap-fit will be descripted below with respect to the decapping of the syringe unit 2. The cap 10 includes in its inside a sleeve-shaped holder 12 (RNS remover) connectable to the rigid needle shield (RNS) 17 of the syringe 15 such that the RNS 17 is removed with the cap 10 if the cap is released from the needle cover sleeve 20. The holder 12 is made of an elastomer and is shrunken onto the RNS during assembly of the syringe unit. As an outer cap housing is made of plastics the holder 12 and the outer cap housing from two-component injection molded part. The cap 10 with the holder are descripted in the patent application EP23165348.6 (Ypsomed) in detail which is incorporated herein by reference. In this embodiment the syringe unit without syringe is devoid of any metal part.

As an alternative the holder can be made of metal and coaxially arranged to the outer cap housing as the holder 12 provided herein. In this case the metal holder engages the RNS 17 and the RNS is removed together with the cap 10 if the cap is released from the needle cover.

The syringe 15 includes the syringe barrel and inside the barrel a movable piston 16 (FIG. 6a). The injection needle 18 (FIG. 6a) is non-releasably connected to a distal end of the syringe barrel. In an unused state the injection needle 18 is covered by the RNS 17, see FIG. 6a. The latter in turn is axially and radially fixedly connected to the cap 10 by the holder 12 as described above once the syringe 15 is mounted to the syringe holder 30.

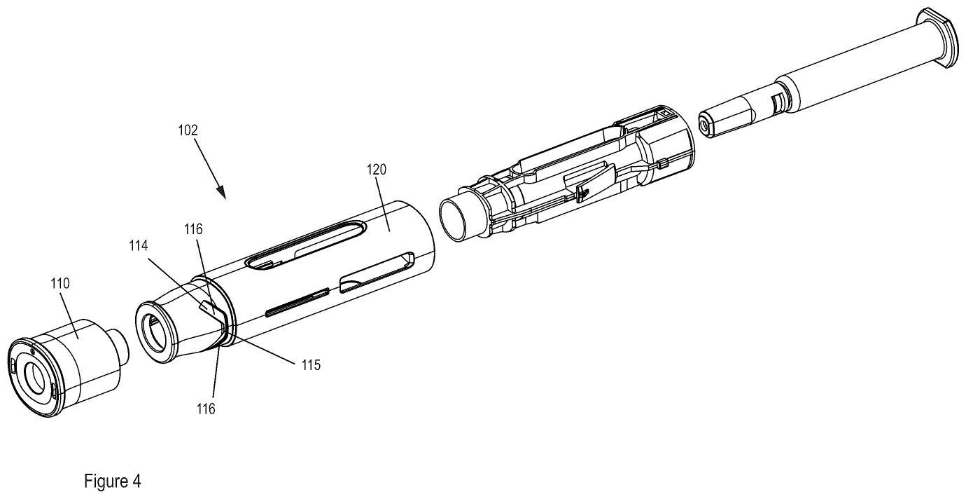

FIG. 4 depicts an alternative syringe unit 102. In contrast to the syringe unit described with respect to FIGS. 2 and 3 the syringe unit 102 shown in FIG. 4 includes a cap 110 mounted to the cover sleeve 120 by a bayonet-like connection.

Instead of the tongues the cap 110 includes two oppositely arranged and radially inward protruding cams or projections (not shown) adapted to project into corresponding grooves 114 in a distal cover sleeve portion.

The grooves 114 are U-shaped. Each groove 114 include a straight groove portion 115 which is perpendicular to the longitudinal axis and on both ends of the straight groove end groove portion 116 are arranged which are angled to the longitudinal axis.

The 110 cap can be mounted and released from the cover sleeve 120, respectively, by a combination of an axial and rotational movement. Hence, in contrast to the embodiment above with the rocker the cap 110 shown in FIG. 4 can be removed from the cover sleeve manually by a user force any time without a release mechanism with a rocker as described above.

If the user grabs the cap 110 and rotates the cap the cam enters the angled groove portions 116 and the cap 110 additionally moves axially in the distal direction away from the cover sleeve end due to the angled groove portions 116. The user can then completely remove the cap 110 from the cover sleeve 120. All other features and functions of the syringe unit are the same as provided herein with respect to the first embodiment shown in FIG. 3.

### Drive Unit

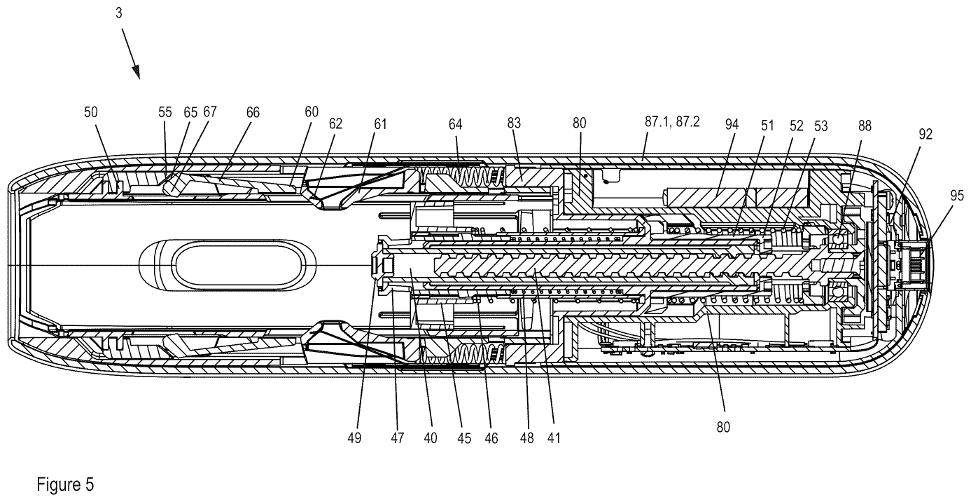

FIG. 5 depicts a cross-sectional view of the drive unit 3 without syringe unit. The cut of the sectional view runs along the longitudinal axis of the drive unit. The drive unit 3 includes a support structure 80 including an outer housing cover or shell 87.1, 87.2 and inside the cover a mechanics sleeve 83 is fixedly connected to the support structure 80. Furthermore, the drive unit 3 includes the opening on its distal side adapted to accommodate a proximal portion of the syringe unit 2.

The support structure 80 guides a gripper sleeve 60 is immovably arranged. Coaxially and inside the gripper sleeve 60 a trigger sleeve 50 is arranged which is movable relative to the supporting structure 80 along the longitudinal axis. Again, inside the trigger sleeve 50 a movable cover sleeve connector 45 is located and biased in the distal direction by a cover sleeve spring 46. The spring abuts on its distal end the cover sleeve connector 45 and on its proximal end a radial wall of the mechanics sleeve 83. A movable sleeve-shaped syringe connector 47 is coaxially arranged inside the cover sleeve connector 45 and biased distally by a syringe connector spring 48 coaxially inside the cover sleeve spring 46 and proximally supported by the mechanics sleeve 83. The spring 46 ensures that the syringe connector 45 pushes the syringe 15 of an inserted syringe unit 2 in the distal direction.

In a proximal portion the support structure 80 supports and guides a sleeve-shaped trigger sleeve connector 51 movable along the longitudinal axis, a drive assembly with an electric motor (not shown) and a gear (not shown) connecting the motor with a threaded rod 41, a battery 94, the treaded rod 41 and a plunger rod 40 threadedly connected with the threaded rod 41. The support structure 80 further accommodates an electronic module 92 with a controller configured to control the electronic motor and providing information to the user via a display or LED 95 or a communication module (not shown). The trigger sleeve connector 51 is fixedly and immovably connected to the trigger sleeve 50 and distally pressed onto a proximal end of the plunger rod 40 by a trigger sleeve spring 53. The plunger rod 40 is non-rotatably guided by the mechanics sleeve 83 and includes on its distal end a flange 49 adapted to engage the piston 16 (see FIG. 6a) during dispensing.

As shown in FIG. 6b inside the proximal portion of the support structure 80 a detection pin 97 is axially movable by the cover sleeve connector 45. The detection pin 97 includes a cam 98, which can interact with a position sensor implemented by two mechanical switches 96.1, 96.2 (FIG. 6b) arranged along the longitudinal axis. The presence of a syringe unit 2 inside the drive unit 3 is thus detectable via detection pin 97 and the first switch 96.1 which provides a corresponding signal to the controller of the electronic module. Furthermore, a retraction of the cover sleeve 20 can be detected by the second switch 96.2 as the cover sleeve 20 retraction further moves the pin 97 with its cam 98 in the proximal direction.

Additionally, in a distal end portion a mechanical switch (not shown) held by the support structure 80 to sense the presence or absence of the cap 10 of an attached syringe unit 2.

The gripper sleeve 60 includes two axially extending and deflectable clamping arms 61 having a clamp 62 at their distal free end adapted to accommodate the syringe holder ledges 33. The clamping arms 61 can be deflected radially outwards allowing the clamps 62 to snap onto the ledges 33 or to release the ledges 33. Furthermore, each clamping arm 61 provides a support for an axially aligned gripper sleeve spring 64 which biases the clamping arms 61 in a holding position.

Each clamping arm 61 includes a mechanical sensor with an actuation rod to detect when the syringe holder ledges 33 are inserted in the clamps 62. Alternatively, a strain gauge or a piezoelectric strain gauge may be arranged on a radial outer surface of the clamping arm 61 and electrically connected to the controller in the electronic module. Based on a strain gauge signal or piezoelectric sensor signal a deflection of the clamping arms 61 can be detected. This allows a determination of whether the clamping arms 61 were pivoted by the ledges 33 during insertion of the syringe unit 2 into the drive unit 3. That in turn allows a determination of whether a syringe unit is completely inserted into and attached to the drive unit.

As a further alternative light guides (fiber optics) may be used to detect the clamping arm deflection. That means a light guide is arranged from the PCB to each clamping arm 61 such that a deflection of the clamping arms 61 interrupts the light guide. The corresponding signal allows a determination of whether the clamping arms were deflected by the ledge 33 of the inserted syringe unit 2.

The gripper sleeve 60 further includes two oppositely arranged and radially inward deflectable actuation arms 66. The actuation arms 66 extend essentially in the longitudinal axis and include on their distal free end 67 a guiding surface 65 or sloped contact surface. The actuation arms 66 are deflectable radially inwards upon contact with a counter guiding surface 55 or counter sloped contact surface of the trigger sleeve 50. The free ends 67 of the actuation arms 66 each further include a radially inward directed contact surface to abut the end bulge 31 of the release arm 32 of the syringe unit 2 as described herein.

The trigger sleeve 50 is axially guided by support structure 80 and shiftable relative thereto. For this purpose, the trigger sleeve 50 includes longitudinal guiding rails (not shown) engaging longitudinal grooves (not shown) in support structure. As shown in FIGS. 5 and 6a the trigger sleeve 50 incudes oppositely arranged contact elements featuring the counter guiding surface 55 or counter sloped contact surface. As provided herein the trigger sleeve 50 is further immovably connected to the trigger sleeve connector 51. As the trigger sleeve connector 51 is biased by the trigger sleeve spring 53 the trigger sleeve 50 is also biased in the distal direction. The trigger sleeve connector 51 in turn is connected to the plunger rod via ledges 52 which engage a proximal shoulder of the plunger rod 40 such that an axial movement of the plunger rod 40 in the proximal direction also moves the trigger sleeve 50 proximally.

In the state shown in FIG. 5 the drive unit 3 is ready to be loaded with a syringe unit 2. The controller displays the current state to the user by means of a steady green light.

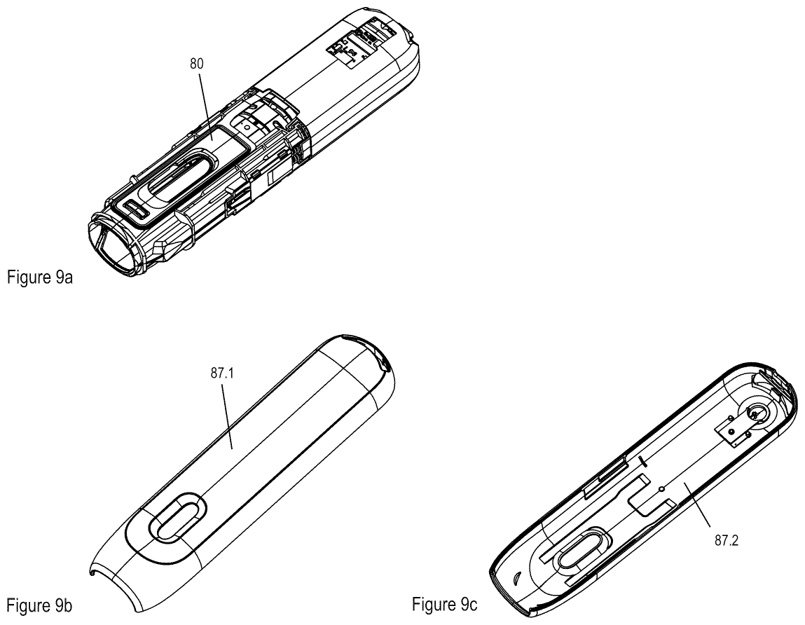

The support structure 80 supports and holds the whole drive mechanism including the afore-mentioned trigger sleeve 50 and gripper sleeve 60 as well as the motor and the electronic module in a proximal portion. The drive unit 3 further includes an outermost shell including two shell parts 87.1, 87.2 that are connected to each other by a snap-fit connection so that the parts are tightly fixed to each other. The support structure with the drive mechanism is depicted in FIG. 9a whereas the upper shell part 87.1 is shown in FIG. 9b and the lower shell part 87.2 in FIG. 9c.

The shell parts do not support any internal component but are fixedly connected to the support structure 80 and act as a grip for the user. The shell parts 87.1, 87.2 may be adjusted in color, in the form, size and appearance for a specific use case.

### Function

In order to prepare the autoinjector 1 for an injection the user attaches a syringe unit 2 to the drive unit 3. The electronics in the drive unit 3 are switched from an inactive state or sleep mode to an active mode upon detection of the syringe unit by the switch 96.1 (FIG. 6b) of the position sensor. Alternatively, the user can press the button 4 to activate the electronics.

FIGS. 6a, 6b and 6c depict cross-sectional views of the drive unit 3 with an inserted syringe unit 2. In FIG. 6a the cut for the cross-sectional view runs along the longitudinal axis in a center plane. The plane of the section for FIGS. 6b and 6c is parallel but in a radial distance to a center longitudinal axis and hence offset from and parallel to the center plane.

In the state shown in FIGS. 6a, 6b and 6c the syringe unit 2 has been inserted with its proximal end portion into the distal opening of the drive unit 3. In the state shown in FIGS. 6a, 6b and 6c the proximal ledges or protrusions 33 of the syringe holder 30 are snapped into the clamps 62 of the gripper sleeve clamping arms 61. The syringe unit 2 is thus held in the drive unit 3 by the clamping arms 61. However, in the state in FIGS. 6a, 6b and 6c the syringe unit 2 can still be pulled out of the drive unit 3 by overcoming a holding force. Hence, if the user pulls the syringe unit 2 in a distal direction the clamping arms 61 are deflected radially. The protrusions 33 of the syringe holder 30 are therefore released from the clamps 62 and the syringe unit 2 can be removed from the drive unit 3.

The presence of the protrusions 33 in the clamps is detected by the controller as the ledges abut a cam of the mechanical switch inside the clamps 62. The controller thus receives a signal that a syringe unit is correctly and completely inserted into the drive unit. Alternatively, if the detection is provided by a strain gauge on the clamping arms 61 or a light guide interrupted by the deflecting clamping arms 61 an engagement movement of the clamping arms is detected and hence the controller can determine that a syringe unit 2 is attached based on the strain gauge or light guide signal.

That means in this state the syringe unit 2 is held inside the drive unit 3 but not yet locked inside the drive unit 3. The mechanical switch in the distal end portion of the support structure 80 is actuated by the cap 10 of the inserted syringe unit. Hence, the controller can determine that a cap 10 is mounted on the inserted syringe unit 2.

In the initial state or shipping state the cover sleeve 20 is locked into the covering position. This is shown in FIG. 6c where the plane for the cross-sectional view is offset a center axis. As shown in FIG. 6a a distally oriented end of the release arm 32 abuts or engages a proximally oriented stop surface 29 of the cover sleeve 20. The release arm 32 is in its locking position. A force in the proximal direction acting on the cover sleeve 20 is thus absorbed by the release arm 32 and hence the cover sleeve cannot be moved out of the covering position towards the retracted position.

Furthermore, in an initial state or shipping state of the syringe unit the syringe barrel does not abut the bearing surface 19 and hence between a distal end of the syringe barrel and the bearing surface 19 of the syringe holder 30 is a gap. The gap may be several millimeters, in particular 2 to 5 mm.

The reason for the presence of the gap is that the device cap 10 has to be movable a short distance if the user grabs the cap 10 to remove the (unused) syringe unit or to position the syringe unit. The travel of the cap is due to a play provided between the parts to account for manufacturing or assembly tolerances. During such a cap movement a removal of the RNS 17 and thus a removal of the sterile barrier is not desired.

As there is an initial gap between the syringe barrel and the bearing surface 19 the RNS (with the needle) can travel a short distance without impact to the sterile barrier. That means the gap between the syringe barrel and the syringe holder bearing surface 19 is, for example, larger than a maximal play of the cap (cap travel relative to the syringe holder).

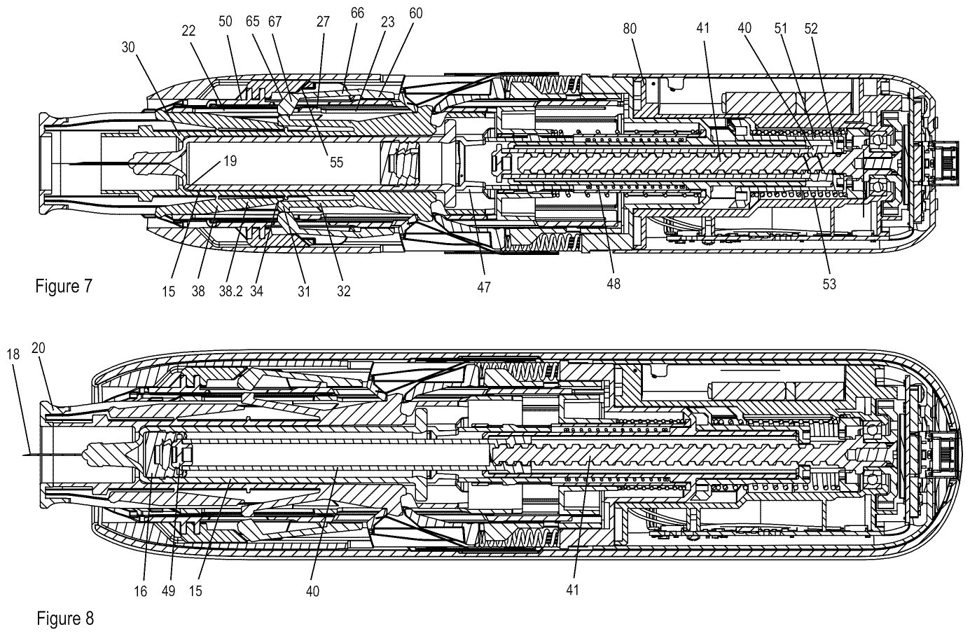

FIG. 7 depicts the autoinjector 1 in a locked state without cap. When the syringe unit 2 is inserted as described herein the proximal end of the syringe unit 2 moves the detection pin 97 proximally and thus the switch 96.1 of the position sensor automatically sends a signal to the controller in the electronic module which detects the presence of the syringe unit 2 inside the drive unit 3. The controller wakes up the electronics and sets the device in an active state. A code reader (not shown) in form of an NFC reader inside the drive unit 3 reads the RFID code 26 of the syringe unit 2. Optionally, a temperature sensor is additionally integrated in the syringe unit RFID code configured to sense the current syringe unit temperature. Subsequently, the controller of the drive unit sends the read medication information and eventually the temperature information from the read code 26 to an external device to verify the drug or medication (and eventually the temperature information) with predefined values or tables. Alternatively, the controller compares the read information from the code 26 with predefined information stored in a memory inside the drive unit 2.

In case of successful medication and eventually temperature verification the controller receives an enable signal (either from the external device or from the internal comparison). The controller then controls the electric motor to rotate the threaded rod 41 to move the trigger sleeve 50 a short distance of several millimeters in the proximal direction. Such a trigger sleeve 50 movement is achieved by a kinematic chain as described as follows.

The motor rotates a pinion and the gear transfers the rotation to the threaded rod 41 which in turn moves the non-rotating plunger rod 40 in the proximal direction, for example, an axial travel of about 5 mm. As the trigger sleeve connector 51 is connected via its ledges 52 to a proximal shoulder of the plunger rod 40 the latter causes the trigger sleeve connector 51 to move and thus the trigger sleeve 50 is shifted in the proximal direction too. Furthermore, the trigger sleeve spring 53 is compressed during the proximal movement.

The proximal movement of the trigger sleeve 50 brings the counter sloped guiding surfaces 55 of the trigger sleeve 50 into contact with the sloped guiding surfaces 65 of the actuation arms. As the two sloped surfaces 65, 55 slide along each other the actuation arms 66 are forced to deflect radially inwards and the actuation arms free ends 67 are pressed radially under the trigger sleeve part with the guiding surface 65 as shown in FIG. 7 when the trigger sleeve 50 is further moved in the proximal direction. The radial inward directed contact surface of the free ends 67 of the deflected actuation arms in turn encounter the end bulges 31 of the release arms 32 thereby deflect the release arms 32 radially inwards too. Subsequently, the inwardly moving release arms 32 abut the rocker actuation surface 34 and tilt the rocker 38 from the first tilt position towards the second tilt position such that the proximal portion 38.2 of the rocker with the actuation surface 34 is pivoted radially inwards.

Consequently, the distal portion 38.1 of the rocker moves radially outwards and hence deflects the holding tongues 11 of the cap 10 radially outwards. That in turn means that the snap-fit connection holding the cap 10 onto the distal end of the cover sleeve 20 is released as the recesses 13 of deflected holding tongues 11 no longer engage the protrusions 28 on the cover sleeve 20. The user can thus pull off the cap 10 form the autoinjector.

When the cap 10 is removed the cam of the mechanical switch is no longer actuated and hence the controller derives from the corresponding switch signal that the cap 10 is removed. Alternatively, if the sensor is implemented with a light guide the removal of the cap 10 interrupts the light guide or alternatively allows an uninterrupted light guide and hence a corresponding signal indicates that the cap is no longer present. Additionally, in case a light guide is used the cap can be illuminated by the light guide if the cap is made of a translucent or transparent plastic. That means the controller may activate a light flashing or constantly illuminating the cap in a color signaling the user that the cap is released and can be removed. If the cap is removed the light guide is interrupted and a cap removal signal is generated for the controller.

If the cap 10 is mounted the syringe 15 inside the syringe holder 30 is hold in place by the cap 10 via holder 12 and RNS 17 and thus the distally biased syringe connector 47 cannot move the syringe 15 relative to the syringe holder 30. However, when the cap 10 is removed the syringe 15 is no longer hold and due to the syringe connector spring 48 the syringe connector 47 moves the syringe 15 relative to the syringe holder 30 distally and thus the gap between the distal end of the syringe barrel and the bearing surface 19 disappears. That means upon cap removal the syringe connector urges the syringe 15 against the bearing surface 19 such that the syringe 15 is fixedly hold between the biased syringe connector 47 and the bearing surface 19.

The radially inwardly deflected release arms 32 have further the effect that they no longer engage the stop surface 29 (FIG. 6c) of the cover sleeve 20 and thus allow for the cover sleeve 20 to move out of a covering position and move inside the support structure 80 in the proximal direction relative to the syringe holder 30.

At the same time the radially inwardly deflected actuation arms 66, namely a proximally oriented surface of the free ends 67 of the actuation arms 66 abut a rib 27 of the cover sleeve 20. The rib 27 is located between the opening 22 and opening 23 in the cover sleeve shown in FIG. 3 and in the cross-sectional view in FIG. 6c. That means in this locked state the syringe unit 2 cannot be pulled out as the free end 67 abuts the rib 27 of the cover sleeve and hence the syringe unit 2 is locked in the drive unit.

The cap 10 of the autoinjector 1 is now removed and the autoinjector is ready for an injection. The controller displays a corresponding notification on the display the LED 95 constantly lights.

As a next step the user places the distal end of the cover sleeve 20 onto an injection site and pushes the autoinjector 1 towards the injection site. This causes the cover sleeve 20 to move proximally from the distal covering position into support structure 80 (push on skin) and compresses the cover sleeve spring 46 which biases the cover sleeve 20 in the distal direction via cover sleeve connector 45. The retraction of the cover sleeve 20 is possible as the end ledges 31 of the release arms 32 are hold radially inwards by the deflected actuation arms 66 (FIG. 7). During its proximal movement the cover sleeve 20 is guided by the guiding elements 91.1, 91.2 which travel inside the guiding grooves in the drive unit. When the cover sleeve 20 is in its retracted position the injection needle 18 protrudes from the cover sleeve as shown in FIG. 8.

Upon push on skin the cover sleeve connector 45 is moved proximally and thus the detection pin 97 is further moved proximally. The further movement brings the cam 98 in contact with the second switch 96.2 (FIG. 6b) which triggers the controller in the electronic module to start the injection. That means the controller drives the electric motor in the dispensing direction and thus rotates the threaded rod 41 and thereby moves the plunger rod 40 in the distal direction.

The controller drives the plunger rod distally with a low torque. Upon contact of the plunger rod flange 49 with the piston 16 inside the syringe barrel the motor current increases due to the motor controlling. That is a signal for the controller that the plunger rod 40 is correctly positioned. Subsequently, the motor drives the plunger rod to dispense the predefined dose.

During dispensing the distal flange 49 of the plunger rod 40 moves the piston 16 inside the syringe 15 in the distal direction and thus dispenses liquid drug through the injection needle out of the syringe 15. If the piston 16 abuts a distal end inside the syringe barrel the current of the motor controlling increases and the controller stops to drive the plunger rod distally. This end of injection condition is shown in FIG. 8. As shown the plunger rod 40 and thus the piston 16 inside the syringe barrel is in a distal end position.

The distal movement of the plunger rod 40 moves the trigger sleeve 50 via trigger sleeve connector 51 in the distal direction to its initial position. That means the actuation arms 66 can move radially outwards back into their non-deflected position. However, as the cover sleeve 20 is in its retracted proximal position the release arms 32 are prevented from being moved back into the initial non-deflected position by the side support surfaces 37 which abut on an inner surface of the retracted cover sleeve. That means as long as the cover sleeve 20 is in its retracted position the release arms 32 remain deflected.

The user may interrupt the dispensing movement by pressing the button 4. The controller stops the motor and thus the plunger rod movement. The dispensing can be continued when the button 4 is pressed again.

The controller displays a holding time indicating a period during that the user has to hold the autoinjector 1 onto the injection site after the injection to ensure that the drug is completely administered and absorbed. The controller may display a down counter informing the user about the remaining holding time. The counter is implemented by a progression bar provided by a display visible from outside. Furthermore, the controller can drive the motor without moving the piston rod at idle speed to generate a motor sound which should prevent the user to remove the autoinjector from the injection site too early.

Once the holding time is elapsed a notification in form of a sound is emitted, indicating the user that the autoinjector can be removed from the injection site. Alternatively, the end of the holding time may be indicated only by a LED.

When the user removes the autoinjector 1 from the injection site compressed cover sleeve spring 46 can release and thus it can move the cover sleeve 20 back distally into the needle covering position.

Upon movement of the cover sleeve 20 into its covering position the side support surfaces 37 get out of contact with the inner surface of the cover sleeve 20 and the release arms 32 can pivot radially outwards back into their locking position in which the free end of the arms 32 abut the contact surface inside the cover sleeve 20 thereby locking the cover sleeve 20 in its covering position. Hence, after the injection process the cover sleeve 20 cannot be moved into the retracted position again.

Upon completion of the injection the controller sends recorded injection data with a time stamp to an external receiver, e.g. a cloud server.

Furthermore, as the actuation arms 66 are not deflected anymore and thus do not contact the distal stop surface of the syringe holder 30 the entire syringe unit 2 can be pulled out of the drive unit by the user. The user can now discard the syringe unit 2 and the drive unit 3 is ready to be loaded with a new syringe unit.

Additionally, or alternatively, the autoinjector can indicate a mis-use by the user. For example, the mis-use may be that the user removes the autoinjector 1 from the skin too early or before the full dose has been dispensed or delivered.

The autoinjector can have a sensor that detects contact with and/or removal from the skin. The autoinjector 1 can have an additional sensor for this purpose or a built-in sensor can perform this function. For example, a skin sensor (not shown) can be provided on the needle cover sleeve 20 of the syringe unit 2.

If the user removes the autoinjector 1 from the skin before the end of the dispensing or delivery process, the cover sleeve 20 can be moved back distally into the needle covering position by the compressed cover sleeve spring 46. As described above, the needle cover sleeve 20 is locked in needle covering position.

The skin sensor (not shown) detects that the user has removed the autoinjector 1 from the skin and provides a corresponding signal to the controller of the electronic module 92. The controller stops to drive the plunger rod 40 distally. In addition, the motor controlling does not detect an increase in motor current.

The controller can thus provide information to the users via a display or via a communication module (not shown) that the user may continue the dispensing or delivery process as the user has only made a partial dispensing or partial delivery of medication. Information is shown on the display or on a communication module (not shown) on how the dispensing or delivery process can be continued.

Partial dispensing or partial delivery of medication means that the user or patient receives too little medication, which endangers their health, and the syringe unit with a partially filled syringe must be thrown away, which causes more waste and medication consumption or higher healthcare costs.

The display or a communication module display how the lock element 32 of the cover sleeve 20 can be released. For example, a code can be entered on the display or on a communication module (not shown), the button can be activated or depressed or the lock element 32 can be unlocked by a doctor or a health care professional via an external receiver, e.g. a cloud.

The lock element 32 can thus be moved in the unlocking position in which it does not engage the needle cover sleeve 20 such that the needle cover sleeve 20 can moved out of the covering position. The controller continues to drive the plunger rod 40 distally. Consequently, the dose remaining in the syringe is administered.

Upon completion of the injection the controller sends information to the users via a display or a communication module or to an external receiver, e.g. a cloud server that the distribution was complete.

While the invention has been described in detail in the drawings and foregoing description, such description is to be considered illustrative or exemplary and not restrictive. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality. The mere fact that certain elements or steps are recited in distinct claims does not indicate that a combination of these elements or steps cannot be used to advantage, specifically, in addition to the actual claim dependency, any further meaningful claim combination shall be considered disclosed.

In the claims, the word “comprising” does not exclude other elements or steps, and the indefinite article “a” or “an” does not exclude a plurality. For example, “an arm” does not exclude the fact that there may be two arms that functionally or structurally fulfill the purpose of “an arm”. The mere fact that certain elements or steps are recited in distinct claims shall not preclude the existence of further meaningful combinations of these elements or steps.

LIST OF DESIGNATIONS

| | |
|---|---|
| 1 | autoinjector |
| 2 | syringe unit |
| 3 | drive unit |
| 4 | button |
| 10 | cap |
| 11 | holding tongue |
| 12 | holder |
| 13 | recess |
| 15 | prefilled syringe |
| 16 | piston |
| 17 | rigid needle shield RNS |
| 18 | injection needle |
| 19 | bearing surface |
| 20 | needle cover sleeve |
| 21 | opening |
| 22 | locking opening |
| 23 | 159opening |
| 24 | medicament window |
| 25 | label |
| 26 | RFID tag |
| 27 | rib |
| 28 | protrusion |
| 29 | stop surface |
| 30 | syringe holder |
| 31 | end bulge |
| 32 | release arm |
| 33 | protrusion/end ledge |
| 34 | actuation surface |
| 35 | center |
| 36 | nose |
| 37 | side support surface |
| 38.1 | distal portion |
| 38 | rocker |
| 38.2 | proximal portion |
| 40 | plunger rod |
| 41 | threaded rod |
| 45 | cover sleeve connector |
| 46 | cover sleeve spring |
| 47 | syringe connector |
| 48 | syringe connector spring |
| 49 | flange |
| 50 | trigger sleeve |
| 51 | trigger sleeve connector |
| 52 | ledge |
| 53 | trigger sleeve spring |
| 55 | counter sloped guiding surface |
| 60 | gripper sleeve |
| 61 | clamping arms |
| 62 | clamp |
| 64 | gripper sleeve spring |
| 65 | sloped guiding surface |
| 66 | actuation arm |
| 67 | free end |
| 80 | support structure |
| 83 | mechanics sleeve |
| 87.1 | first shell |
| 87.2 | second shell |
| 88 | bearing |
| 91.1 | first guiding element |
| 91.2 | second guiding element |
| 92 | electronics |
| 94 | battery |
| 95 | LED |
| 96.1 | first watch |
| 96.2 | second switch |
| 97 | pin |
| 98 | cam |
| 102 | syringe unit |
| 110 | cap |
| 114 | groove |
| 115 | straight portion |
| 116 | angled portion |
| 120 | cover sleeve |

What is claimed is:

1. A disposable reservoir unit for an injection device for dispensing a liquid drug through an injection needle, the reservoir unit being configured for releasable attachment to a reusable drive unit of the injection device, the reservoir unit comprising:
a reservoir holder for holding a reservoir in place and comprising guiding means and defining a longitudinal axis;
two radially extending and oppositely arranged protrusions for releasably attaching the reservoir unit to the drive unit; and
a needle cover sleeve guided by the guiding means and movable along the longitudinal axis relative to the reservoir holder between a covering position in which the injection needle is covered and a retracted position in which the injection needle protrudes from the needle cover sleeve,
wherein the needle cover sleeve comprises two oppositely arranged lateral locking openings adapted to accommodate actuation arms of the drive unit to lock the reservoir unit to the drive unit,
wherein in a plane perpendicular to the longitudinal axis, the needle cover sleeve comprises a first outer dimension along a first axis and a second outer dimension along a second axis perpendicular to the first axis, wherein the first outer dimension is larger than the second outer dimension and wherein the protrusions are arranged on the first axis,
wherein the needle cover sleeve comprises two oppositely arranged pairs of guiding elements protruding from an outer surface of the needle cover sleeve and adapted to engage counter guiding elements in the drive unit to prevent rotation of the reservoir unit relative to the drive unit,
wherein a first guiding element of each pair is arranged on a first side and a second guiding element of each pair is arranged on a second side of the first axis in the plane perpendicular to the longitudinal axis, and
wherein the second outer dimension is between 2 and 3.6 times larger than a distance from a center of the first guiding element to a center of the second guiding element of each pair in the plane perpendicular to the longitudinal axis.

2. The disposable reservoir unit according to claim 1, wherein in a plane perpendicular to the longitudinal axis the guiding elements protrude along an axis which is angled to the first axis in an angle between 0° and 45°.

3. The disposable reservoir unit according to claim 1, wherein the first outer dimension is between 1.05 and 1.5 times larger than the second outer dimension.

4. The disposable reservoir unit according to claim 1, wherein the needle cover sleeve is an outermost part of the reservoir unit and the reservoir holder is concentrically arranged inside the needle cover sleeve.

5. The disposable reservoir unit according to claim 1, wherein in a plane perpendicular to the longitudinal axis an outer form of the reservoir unit is a regular oval form, which extends along a length of the reservoir unit in a longitudinal direction.

6. The disposable reservoir unit according to claim 1, wherein each protrusion is arranged adjacent to a respective locking opening with respect to a longitudinal direction.

7. The disposable reservoir unit according to claim 1, wherein the locking openings are located distally from the protrusions.

8. The disposable reservoir unit according to claim 1, wherein the locking openings extend along the longitudinal axis such that the needle cover sleeve is not hindered and can be moved relative to the protrusions between the covering position and the retracted position when the actuation arms are positioned inside the locking openings.

9. The disposable reservoir unit according to claim 1, wherein the protrusions comprise a rectangular-shaped cross section in a plane parallel to the longitudinal axis.

10. The disposable reservoir unit according to claim 1, wherein the needle cover sleeve further comprises two oppositely arranged second openings accommodating the protrusions and the protrusions are accessible therethrough, and wherein the second openings extend along the longitudinal axis such that the needle cover sleeve can be moved relative to the protrusions between the covering position and the retracted position without covering the protrusions.

11. The disposable reservoir unit according to claim 1, further comprising a lock element movable relative to the needle cover sleeve, wherein the lock element is configured to be in a locking position in which the lock element engages the needle cover sleeve and prevents the needle cover sleeve from being moved out of the covering position, and the lock element is configured to be in an unlocking position in which the lock element does not engage the needle cover sleeve such that the needle cover sleeve is not prevented from being moved out of the covering position.

12. The disposable reservoir unit according to claim 11, wherein the lock element is a radially protruding flexible arm with a free end portion, wherein the needle cover sleeve comprises a stop surface, and wherein the free end portion is adapted to engage the stop surface to hold the needle cover sleeve in the covering position when the lock element is in the locking position.

13. The disposable reservoir unit according to claim 12, wherein the end portion engages the stop surface in a non-deflected position of the arm and the end portion is disengaged from the stop surface in a deflected state of the arm thereby allowing the needle cover sleeve to be moved out of the covering position.

14. The disposable reservoir unit according to claim 1, wherein the reservoir unit, without the reservoir, is devoid of metallic parts.

15. An injection device comprising the disposable reservoir unit according to claim 1, wherein the reusable drive unit comprises a plunger rod and a drive for moving the plunger rod in a dispensing direction to dispense the drug from the reservoir, wherein the reservoir unit is releasably attachable to the drive unit.
